(19) United States
(12) Patent Application Publication
(10) Pub. No.: US 2025/0009392 A1
(43) Pub. Date: Jan. 9, 2025

(54) ESTHETICIAN'S COMEDONE EXTRACTOR TOOL

(71) Applicant: Douglas Preston Easley, San Jose, CA (US)

(72) Inventor: Douglas Preston Easley, San Jose, CA (US)

(21) Appl. No.: 18/219,587

(22) Filed: Jul. 7, 2023

(51) Int. Cl. A61B 17/50

(52) U.S. Cl. CPC A61B 17/50; A61B 2017/00747

(57) ABSTRACT

Embodiments of the present invention provide a skincare device for debris extraction (e.g., comedone, sebum, dead skin, etc.) from skin pores to improve the health and appearance of the skin. The medical device is an extractor tool that can be used by estheticians, for example, to treat patients in a way that is gentle on the skin and more effective at debris removal than traditional extractor tools. The extractor tool can be used to treat or prevent congestive acne and other skin conditions that result in blackhead and whiteheads, for example.

### FIELD

Embodiments of the present invention generally relate to the field of skincare. More specifically, embodiments relate to tools used for skincare treatment.

### BACKGROUND

In the field of skincare, small pencil-shaped medical tools are often used for treatment, diagnosis, and procedures that improve skin health or that address specific skin conditions. Estheticians that focus on improving skin health and appearance often use these tools to treat their patients.

Comedone sebum refers to the oily plug of sebum and dead skin cells that forms in a dilated hair follicle. The pilosebaceous unit, which includes a hair follicle and sebaceous gland, produces sebum that coats, moisturizes, and protects the skin. Comedones can form when the cells lining the sebaceous duct proliferate leading to increased sebum production. A pore that is completely closed to the surface can result in “whiteheads,” and wide-open pores can oxidize, resulting in “blackheads,” which can be mitigated with appropriate treatment.

Existing medical tools used to treat these skin conditions, often referred to as extractor tools, can cause unnecessary stress to the skin during treatment, and often do not remove all of the debris from the skin when used to treat blackheads and other pore/skin diseases.

FIG. 1 depicts a prior art skin extraction tool 100 that includes a rigid body 115 having a pointed hook-shaped end 105 and a fenestrated or “cup-and-hole” end 110 connected to used to extract debris from skin pores. The pointed hook-shaped end 105 is designed to pierce the skin, which can cause significant trauma to the skin, and is exacerbated by pressure applied using the fenestrated end 110. By pressing on the skin using fenestrated end 110, sebum, dead skin cells, and debris that contribute to the formation of blackheads and whiteheads may be removed. However, the use of these tools often results in irritation, bruising, and other unwanted skin conditions, without removing the majority of the debris from the pore.

FIGS. 2-4 depict other prior art debris extraction tool tip designs. Specifically, FIG. 2 depicts a tool having a closed-loop or wire-loop end 205 that is similar to fenestrated end 110 depicted in FIG. 1. However, wire-loop end 205 includes a larger open area for use on larger areas of the skin (e.g., to treat larger blackheads, pimples, and other skin conditions). FIG. 3 depicts another tool having a fenestrated end 305 with a smaller opening designed to apply more pressure to a target area of the skin. However, applying more pressure in this way often results in skin irritation and can be painful to the patient. FIG. 4 depicts a tool having a lancet-style end 405 for piercing the skin or lancing a pimple, blackhead, etc., so that the pore can be drained to remove the debris (e.g., sebum). However, as mentioned above, tearing/opening the skin using a lancet can cause significant trauma to the skin. Accordingly, a more effective approach to treating these skin conditions using relatively gentle means is desired.

### SUMMARY

Embodiments of the present invention provide a device for debris extraction (e.g., comedone, sebum, dead skin, etc.) to treat skin pores for improving the health and appearance of the skin. The novel device is an extractor tool that can be used by estheticians, for example, to treat patients in a way that is gentle on the skin and more effective at debris removal than traditional devices. The extractor tool can be used to treat or prevent congestive acne and other skin conditions that result in blackhead and whiteheads, for example.

According to one disclosed embodiment, an extractor tool for removing debris from a pore is disclosed. The extractor tool includes a shaft including a hash pattern texture that facilitates fine movement of a tip, the tip coupled to the shaft, where the tip is of a pointed and substantially triangular shape, and where an end of the tip is curled back towards the shaft forming a rounded curve.

According to some embodiments, the tip is operable to press on skin around the pore to apply pressure to the pore from below.

According to some embodiments, the tip is further operable to press on skin around the pore to extract debris from the pore.

According to some embodiments, the debris includes comedone.

According to some embodiments, the debris includes sebum.

According to some embodiments, the debris includes dead skin.

According to some embodiments, the pore includes a blackhead or whitehead.

According to some embodiments, the shaft and the tip are made from surgical steel.

According to some embodiments, the shaft and the tip are made from stainless steel.

According to another embodiment, a method of extracting debris from a pore is disclosed. The method includes pinching skin around the pore and applying force to the pore from below using an extractor tool. The extractor tool includes a rod-shaped shaft including a hash pattern texture that facilitates fine movement of a pointed tip, and the pointed tip coupled to the shaft, where the pointed tip is curved back towards the shaft.

According to some embodiments, the tip is operable to press on skin around the pore to apply pressure to the pore from below a follicle of the pore.

According to some embodiments, the tip is further operable to press on skin around the pore to extract debris from the pore.

According to some embodiments, the debris includes comedone.

According to some embodiments, the debris includes sebum.

According to some embodiments, the debris includes dead skin.

According to some embodiments, the pore includes a blackhead or whitehead.

According to some embodiments, the shaft and the tip are made from a surgical steel.

According to some embodiments, the shaft and the tip are made from a stainless steel.

### BRIEF DESCRIPTION OF THE DRAWINGS

The accompanying drawings, which are incorporated in and form a part of this specification, illustrate embodiments of the invention and, together with the description, serve to explain the principles of the invention:

FIG. 1 is a diagram depicting a typical skin extractor tool that includes a pointed hook-shaped end and a fenestrated end used to extract debris from skin pores.

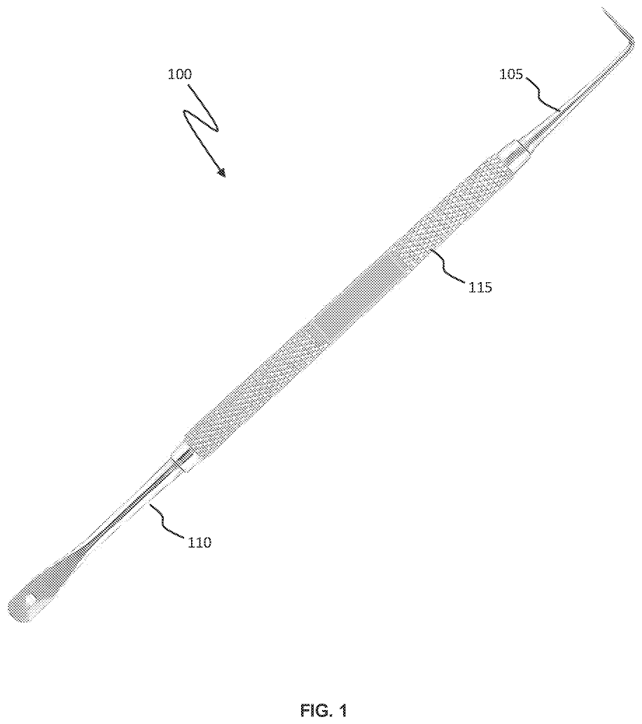

FIG. 2 is a diagram depicting an end of a typical skin extractor tool of the prior art that includes a closed-loop or wire-loop end.

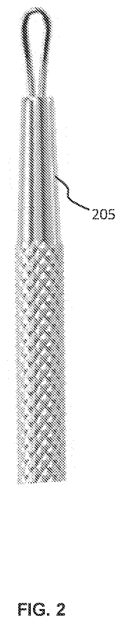

FIG. 3 is a diagram depicting an end of a typical skin extractor tool of the prior art that includes a smaller fenestrated end designed to apply more pressure to a smaller area of the skin.

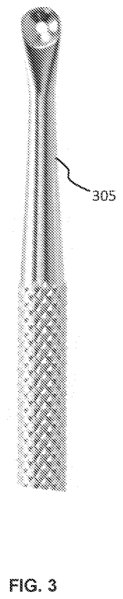

FIG. 4 is a diagram depicting an end of a typical skin extractor tool of the prior art that includes a lancet-style end for piercing the skin or lancing a pimple, blackhead, etc., so that the pore can be drained to remove the debris.

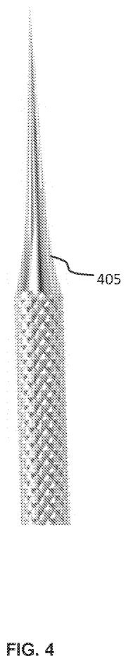

FIG. 5A is a diagram depicting an isometric view of an exemplary extractor tool according to embodiments of the present invention.

FIG. 5B is a diagram depicting a top-down view of an exemplary extractor tool according to embodiments of the present invention.

FIG. 6A is a diagram depicting a close isometric view of an exemplary curved tip of an extractor tool for treating skin and specifically for applying pressure below a follicle to remove debris from the pore without applying significant pressure or tearing the skin according to embodiments of the present invention.

FIG. 6B is a diagram depicting a close top-down view of an exemplary curved tip of an extractor tool according to embodiments of the present invention.

FIG. 6C is a diagram depicting a close top-down view of an exemplary curved tip of an extractor tool according to embodiments of the present invention.

FIG. 7 is a flow chart depicting an exemplary process for removing debris from a pore using an extractor tool according to embodiments of the present invention.

FIG. 8A is a diagram of the first step of an exemplary skin treatment using an extraction tool described herein according to embodiments of the present invention.

FIG. 8B is a diagram of the second step of an exemplary skin treatment using an extraction tool described herein according to embodiments of the present invention.

FIG. 9 is a diagram depicting a close side view of an exemplary curved tip of an extractor tool having a 1.00 mm radius according to embodiments of the present invention.

### DETAILED DESCRIPTION

Reference will now be made in detail to several embodiments. While the subject matter will be described in conjunction with the alternative embodiments, it will be understood that they are not intended to limit the claimed subject matter to these embodiments. On the contrary, the claimed subject matter is intended to cover alternative, modifications, and equivalents, which may be included within the spirit and scope of the claimed subject matter as defined by the appended claims.

Furthermore, in the following detailed description, numerous specific details are set forth in order to provide a thorough understanding of the claimed subject matter. However, it will be recognized by one skilled in the art that embodiments may be practiced without these specific details or with equivalents thereof. In other instances, well-known methods, procedures, components, and circuits have not been described in detail as not to unnecessarily obscure aspects and features of the subject matter.

Portions of the detailed description that follows are presented and discussed in terms of a method. Although steps and sequencing thereof are disclosed in a figure herein describing the operations of this method, such steps and sequencing are exemplary. Embodiments are well suited to performing various other steps or variations of the steps recited in the flowchart of the figure herein, and in a sequence other than that depicted and described herein.

Embodiments of the present invention provide a medical device for debris extraction (e.g., comedone, sebum, dead skin, etc.) from skin pores to improve the health and appearance of the skin. The medical device is an extractor tool that can be used by estheticians, for example, to treat patients in a way that is gentle on the skin and more effective at debris removal than traditional extraction tools. The extractor tool can be used to treat or prevent congestive acne, oily skin, and other skin conditions that result in blackhead and whiteheads, for example.

According to various embodiments, the extractor tool includes a pencil or rod-shaped shaft and an end with a curved tip, unlike a lancet or fenestrated design, that treats pores from underneath in a way that does not cut, tear, or apply significant, targeted pressure to the skin. Rather, the extraction tools described according to embodiments of the present invention apply a smooth, relatively gentle force along the surface of the skin to “cup” skin pores and gently apply pressure to press, scoop and guide debris away from skin pores. Moreover, rather than treating only a single pore or small section of the skin, the improved tip designs described herein can treat a larger area in a continuous motion that is gentler on the skin and more efficient and effective at removing debris from pores. As such, skincare providers are able to treat patients more rapidly, and patients benefits from improved skin health without risks of inflammation, irritation, infection, bruising, etc.

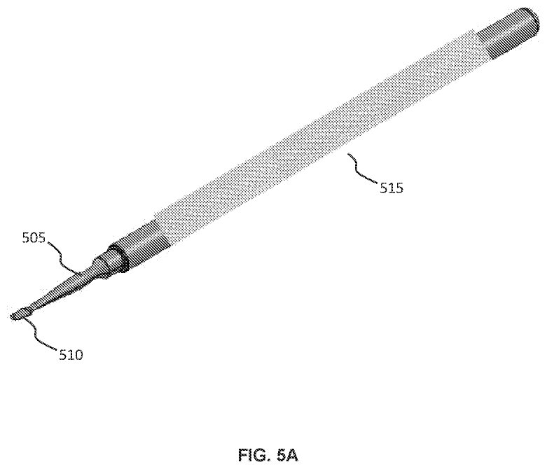

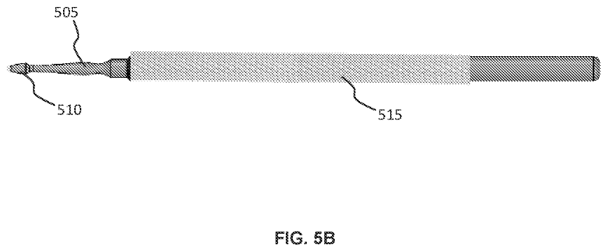

FIG. 5A depicts an isometric view of an exemplary extraction tool 500 according to embodiments of the present invention. FIG. 5B depicts a top-down view of extraction tool 500. Extraction tool 500 includes an end 505 with an upward/inward curving tip 510. Tip 510 does not formed a fully enclosed loop, but forms an open curled or wave-shape with a pointed tip suitable for guiding debris out of skin pores. Rather than cutting or tearing the skin, or applying significant pressure to a relatively small area, tip 510 is operable to apply relatively light, controllable force directed underneath the pore/follicle, so that any debris can be removed from the pore from below, using less pressure compared to existing approaches that apply top-down force or cut and tear the skin. Moreover, tip 510 is operable to treat specific spots on the skin, or can glide/drag over larger skin surfaces, applying pressure from below the follicles to remove substantial amounts of debris in a single easy movement, which can be repeated as necessary. In this way, the speed and efficiency of skin treatment is improved without risking inflammation and other unwanted skin conditions resulting from the treatment.

The extractor tool 500 includes a rigid body or shaft 515 typically made from surgical steel, medical-grade steel, or any stainless steel or similar material that can withstand high temperature cleaning using a sanitizing solution, typically a rust-proof metal. According to some embodiments, end 505 is secured to shaft 515 using threaded coupling means so that end 505 can be readily removed and cleaned or replaced with a different tip. Some embodiments can include an end 505 disposed at each end of shaft 515, and the ends can be removeable/replaceable. The ends can include open-ended curved tips of different shapes, sizes, etc. The shaft 515 of extractor tool 500 is typically textured to improve grip and handling, such as a rough hash or diamond pattern finish that prevents slippage.

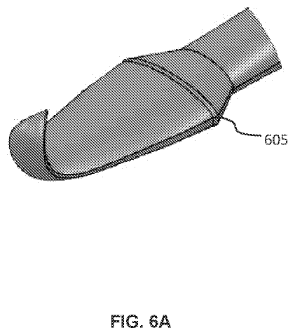

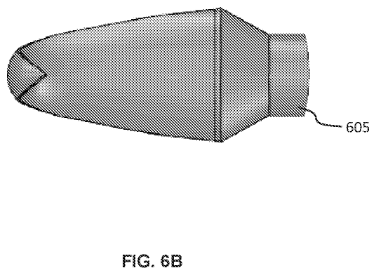

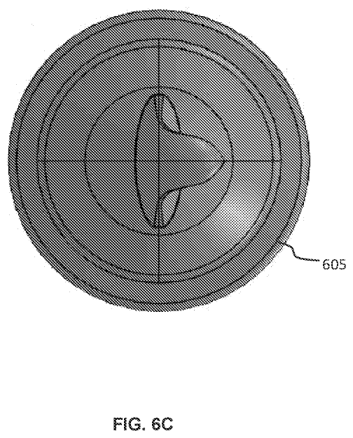

FIG. 6A is a close isometric view of an exemplary curved tip 605 of an extraction tool for treating skin and specifically for applying pressure below a follicle to remove debris from the pore without applying significant pressure or tearing the skin according to embodiments of the present invention. FIG. 6B is a close top-down view of curved tip 605. FIG. 6C is a close top-down view of curved tip 605. Tip 605 can be a component of a removeable or fixed end of an extractor tool, for example. The curved tip 605 can be considered a flat triangular or spade shaped tip that is bent upward at the end and curved back towards the shaft of the extractor. The bent/curved tip 605 functions as a open scoop that can apply pressure from underneath a pore's follicle and can capture the debris excreted from the pore (e.g., comedone, sebum, dead skin, etc.) using gentle force. The skin around the pore can be pinched to increase pressure applied to the pore when using curved tip 605 to dislodge and remove the target debris. In contrast to existing extraction tools, the curved tip 605 is not a closed loop, a fenestrated lancet, or a pointed lancet designed to pierce the skin. Advantageously, the debris can be scooped from the follicle using force form below without using harmful amounts of pressure or causing other trauma to the skin, such as cutting or tearing.

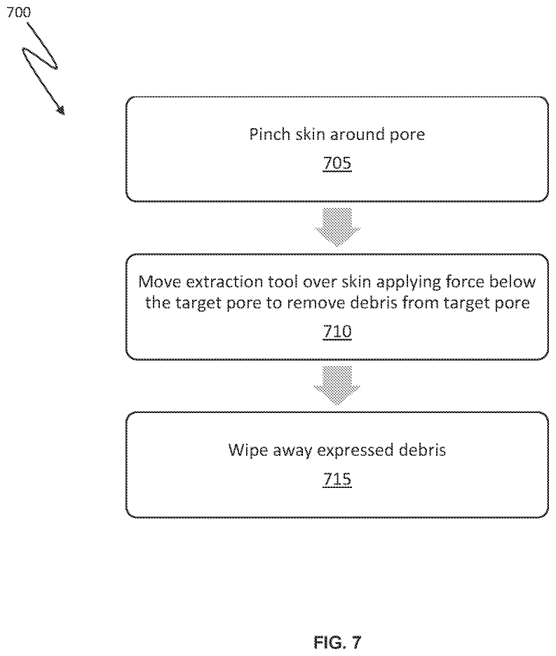

FIG. 7 is a flow chart depicting steps of an exemplary process 700 for removing debris from a pore using an extractor tool according to embodiments of the present invention. The extractor tool can be a pencil or rod-shaped shaft with a curved tip, unlike a lancet or fenestrated design, for treating pores in a way that does not cut, tear, or apply significant, targeted pressure to the skin. As described above, the extractor tool applies a smooth, relatively gentle force along the surface of the skin to “cup” skin pores and gently apply pressure to scoop, press, and guide debris away from skin pores.

At step 705, skin around a target pore is pinched to increase the pressure on the pore.

At step 710, an extractor tool is moved over the skin and/or the pore to apply a force from below the target pore. According to some embodiments, the pressure is applied underneath a follicle of the pore. As a result of the movement of the extractor tool, debris such as comedone is forced out of the pore to cleanse and relieve the poor, which improves overall skin health.

At step 715, the expressed debris is optionally wiped away, and the process can be repeated again beginning at step 705 as necessary.

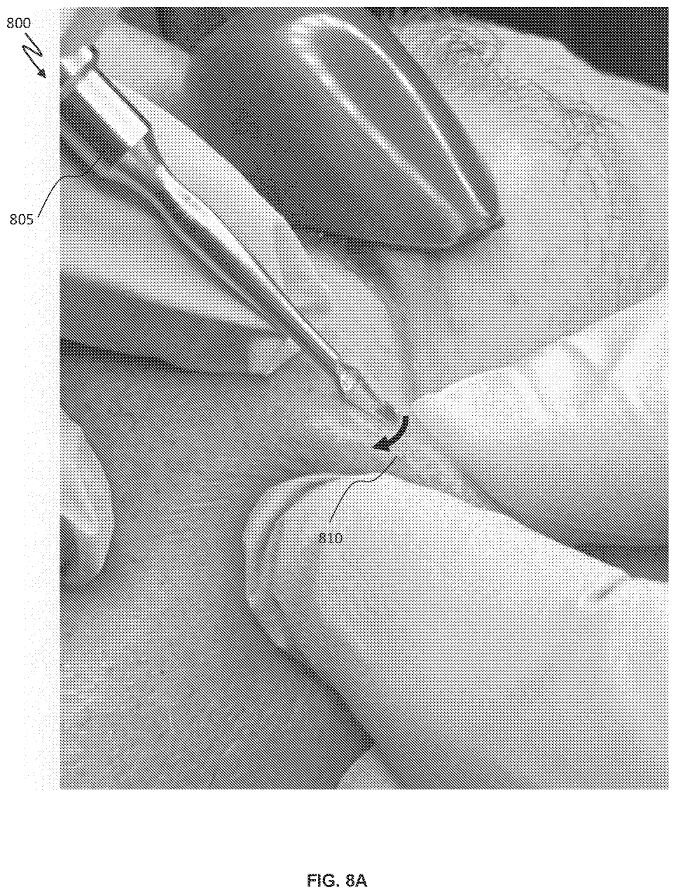

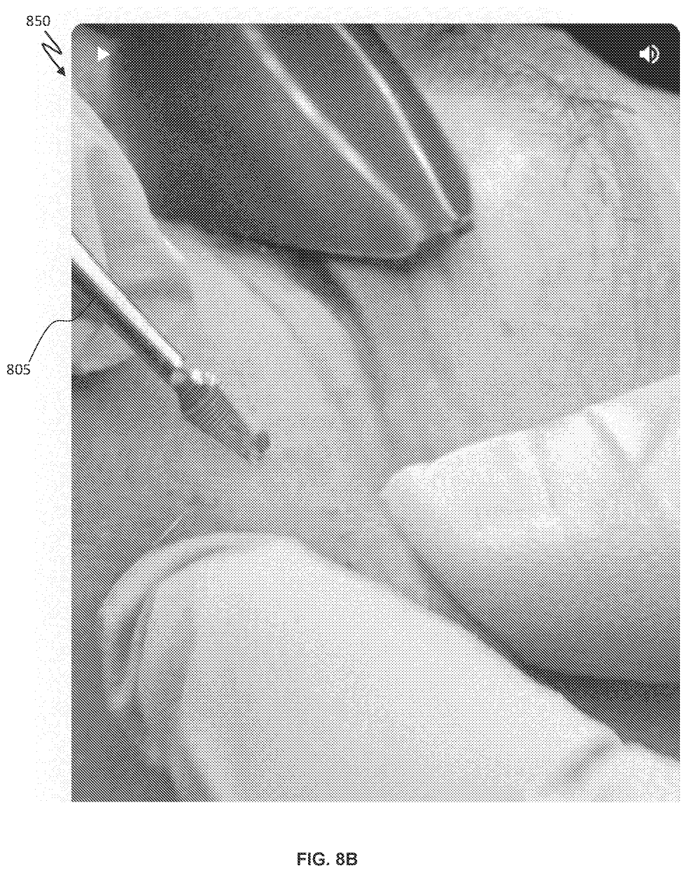

FIG. 8A is a diagram of the first step 800 of an exemplary skin treatment using an extraction tool 805 described herein according to embodiments of the present invention. As depicted in FIG. 8A, the skin 810 around a pore is pinched and the extraction tool 805 is pressed down on the surface of the skin 810 near the pore. FIG. 8B is a diagram of the second step 850 of an exemplary skin treatment using the extraction tool 805 described herein according to embodiments of the present invention. In FIG. 8B, the extraction tool 805 is moved sideways across the surface of the skin to scoop or move the debris out and away from the target pore. The extracted debris accumulates on the tip of the extraction tool 805 until it is wiped away. In this way, the pore is cleansed, and the patient's skin health is improved without significant pressure, bruising, inflammation, etc.

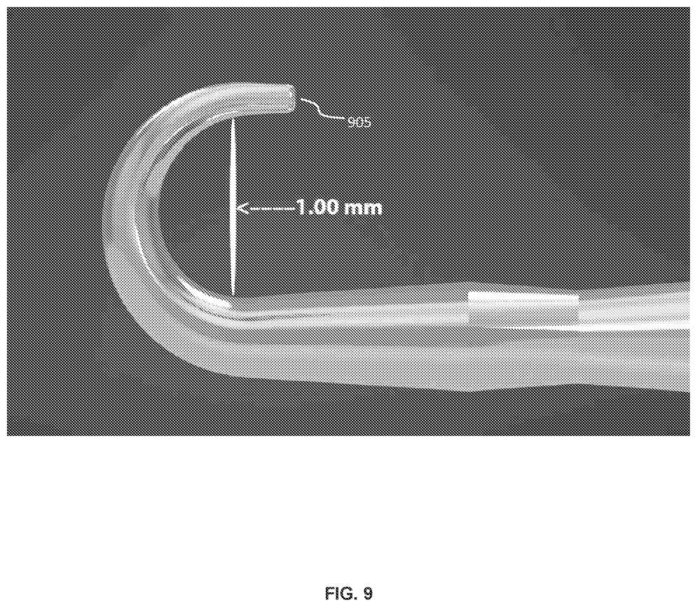

FIG. 9 is a diagram depicting a close side view of an exemplary curved tip 905 of an extractor tool having a 1.00 mm radius according to embodiments of the present invention. The edge of the curved tip is smaller than the radius of the curved tip. According to some embodiments, the edge of the curved tip is 0.52 mm wide.

Embodiments of the present invention are thus described. While the present invention has been described in particular embodiments, it should be appreciated that the present invention should not be construed as limited by such embodiments, but rather construed according to the following claims.

What is claimed is:

1. An extractor tool for removing debris from a pore, the extractor tool comprising:
a shaft comprising a hash pattern texture that facilitates fine movement of a tip; and
the tip coupled to the shaft, wherein the tip is of a pointed and substantially triangular shape, and wherein an end of the tip is curled back towards the shaft forming a rounded curve.

2. The extractor tool of claim 1, wherein the tip is operable to press on skin around the pore to apply pressure to the pore from below.

3. The extractor tool of claim 2, wherein the tip is further operable to press on skin around the pore to extract debris from the pore.

4. The extractor tool of claim 3, wherein the debris comprises comedone.

5. The extractor tool of claim 3, wherein the debris comprises sebum.

6. The extractor tool of claim 3, wherein the debris comprises dead skin.

7. The extractor tool of claim 3, wherein the pore comprises a blackhead or whitehead.

8. The extractor tool of claim 1, wherein the shaft and the tip are made from surgical steel.

9. The extractor tool of claim 1, wherein the shaft and the tip are made from stainless steel.

10. A method of extracting debris from a pore, the method comprising:
pinching skin around the pore; and
applying force to the pore from below using an extractor tool, wherein the extractor tool comprises:
a rod-shaped shaft comprising a hash pattern texture that facilitates fine movement of a pointed tip; and
the pointed tip coupled to the shaft, wherein the pointed tip is curved back towards the shaft.

11. The method of claim 10, wherein the tip is operable to press on skin around the pore to apply pressure to the pore from below a follicle of the pore.

12. The method of claim 11, wherein the tip is further operable to press on skin around the pore to extract debris from the pore.

13. The method of claim 12, wherein the debris comprises comedone.

14. The method of claim 12, wherein the debris comprises sebum.

15. The method of claim 12, wherein the debris comprises dead skin.

16. The method of claim 12, wherein the pore includes a blackhead or whitehead.

17. The method of claim 10, wherein the shaft and the tip are made from a surgical steel.

18. The method of claim 10, wherein the shaft and the tip are made from a stainless steel.
